(19) United States
(12) Patent Application Publication
(10) Pub. No.: US 2012/0129693 A1
(43) Pub. Date: May 24, 2012

(54) SEED HAVING INOCULATED WITH BACTERIAL BILFILM

(76) Inventor: Takashi Ano, Osaka (JP)

(21) Appl. No.: 13/322,045

(22) PCT Filed: Mar. 30, 2010
(86) PCT No.: PCT/JP2010/056128

§ 371 Date: Feb. 7, 2012

(30) Foreign Application Priority Data
May 28, 2009 (JP) 2009 128816

(51) Int. Cl. A01N 25/26; C12N 11/02

(52) U.S. Cl. 504/100; 435/177

(57) ABSTRACT

Disclosed are: a seed having resistance to diseases; and a method for inoculating a microorganism into a seed for the purpose of controlling diseases. As a result of intensive studies, it is found that diseases of a crop can be controlled by inoculating a biofilm of a Bacillus bacterium into a seed of the crop.

Specifically disclosed are; a seed inoculated with a biofilm of a Bacillus bacterium and a method for inoculating a biofilm of a Bacillus bacterium into a seed.

### TECHNICAL FIELD

The present invention relates to a seed having inoculated with a biofilm of a Bacillus bacterium and a method for inoculating a biofilm of a Bacillus bacterium into a seed.

### BACKGROUND

Prevention of damages due to a disease to plants, especially crops, is an important issue. The importance of the issue of controlling diseases of plants is increasing, as it is said recently that excessive use of agricultural chemicals and fertilizers has resulted in on one hand the reduction of soil fertility and on the other hand the emergence of pathogens resistant to those chemicals. To date, methods of prevention of damages due to a disease to plants, such as spraying agricultural chemicals and development of resistant cultivars, have been conducted. However, these methods are not necessarily desirable, because in addition to the fact that a vicious circle as stated above is brought about, food security and environment protection are demanded nowadays.

On the other hand, attempts have been made to control diseases or to improve growth of crops by introducing a microorganism into soil, a seed, root or tuber. Because microorganisms may secret plant nutrients, biological activators and biological disinfectants, it is thought that, upon inoculation into plants, microorganisms provide nutrition, biological activation, biological disinfection and so on to result in the effects as stated above. Prevention of damages due to a disease of plants or improvement of growth by introduction of microorganisms is desirable in terms of safety and environmental protection, and gives preferable images to consumers. Further, it is superior compared to conventional methods using agricultural chemicals or fertilizers, in that it requires less time, costs and labor.

To date, induction of induced systemic resistance (ISR) to a plant by introducing a rizosphere bacterium such as species of Pseudomonas, certain gram negative bacteria and so on into the plant has been reported (Non-Patent Document 1).

Bacillus bacteria exist universally in waters and soils. The properties of Bacillus bacteria have been studied well. Besides, Bacillus bacteria have been used in the production of fermented food and enzymes and have safety and less impact on the environment. Thus, they are desirable as microorganisms to be introduced into plants. Bacillus bacteria are aerobic or facultative anaerobic bacilli which are usually gram positive. Under certain conditions, they form spores trough prespores. Species of Bacillus are known to form highly resistant spores and to produce broad spectrum antibiotics (Non-Patent Document 2). To date, introduction of Bacillus bacteria into a plant has not been reported.

### PRIOR ART DOCUMENTS

### Non-Patent Literature s

Non-Patent Literature 1

Kloepper J W, Ryu C-M, Zhang S., 2004. Induced systemic resistance and promotion of plant growth by Bacillus spp. Phytopathol. 94, 1259-66.

### Non-Patent Literature 2

Cavaglieri, L., Orlando, J., Rodriguez, M. I., Chulze, S., Etcheverry, M., 2005. Biocontrol of Bacillus subtilis against Fusarium verticillioides in vitro and at the maize root level; Res. In Microbiol. 156, 748-754.

### Non-Patent Literature 3

Phae, C. G., Shoda, M., Kuboda, H., 1990. Suppressive effect of Bacillus subtilis and its products on phytopathogenic microorganisms. J. Ferment. Bioeng. 69, 1-7.

### Non-Patent Literature 4

Hiraoka, H., Asaka, O., Ano, T., Shoda, M., 1992. Characterization of Bacillus subtilis RB14, coproducer of peptide antibiotics iturin A and surfactin. J. Gen. Appl. Microbiol. 38, 635-640.

### Non-Patent Literature 5

Rahman M S, Ano T and Shoda M, 2006. Second stage production of iturin A by induced germination of Bacillus subtilis RB14. J. Biotech. 125, 513-515.

### Non-Patent Literature 6

Asaka O, Shoda M, 1996. Biocontrol of Rhizoctonia solani damping off of tomato with Bacillus subtilis RB14. Appl. Environ. Microbiol. 62, 4081-4085.

### Non-Patent Literature 7

Conover R A (1949) Rhizoctonia canker of tomato. Phytopathol. 39, 950-951.

### Non-Patent Literature 8

Pratt, L. A., Kolter, R., 1999. Genetic analyses of bacterial biofilm formation. Curr. Opin. Microbiol. 2, 598-603.

### Non-Patent Literature 9

Branda, S. S., Gonzalez-Pastor, J. E., Ben-Yehuda, S., Losick, R., Kolter, R., 2001. Fruiting body formation by Bacillus subtilis. Proc. Natl. Acad. Sci. 98, 11621-11626.

### Non-Patent Literature 10

Branda, S. S., Gonzalez-Pastor, J. E., Dervyn, E., Ehrlich, S. D., Losick, R., Kolter, R., 2004. Genes involved in formation of structured multicellular communities by Bacillus subtilis. J. Bacteriol. 186, 3970-3979.

### Non-Patent Literature 11

Branda, S. S., Vik, S., Friedman, L., Kolter, R., 2005. Biofilms: the matrix revisited. Trends Microbiol. 13, 20-26.

### SUMMARY OF INVENTION

It is aimed to provide a seed having resistance to damages due to a disease as well as a method for inoculating a microorganism into a seed for the prevention of damages due to a disease.

The present inventors studied intensively and found that damages due to a disease of a crop can be prevented by inoculating a biofilm of a Bacillus bacterium into a seed, to complete the invention. The present invention provides a seed having inoculated with a biofilm of a Bacillus bacterium. The present invention further provides a method for inoculating a biofilm of a Bacillus bacterium into a seed.

### BRIEF DESCRIPTION OF THE DRAWINGS

FIG. 1 represents results of growth inhibition test on plates with R. solani K1 using seeds coated with Bacillus subtilis RB14 BF, seeds coated with liquid shaking culture, or seeds coated with sterilized BF added with RB14 of liquid culture.

FIG. 2 represents results of growth inhibition test on tomato plants with R. solani K1 using seeds coated with Bacillus subtilis RB14 BF, seeds coated with liquid shaking culture, or seeds coated with sterilized BF added with RB14 of liquid culture.

FIG. 3 shows micrographs of Bacillus subtilis RB14 BF of the coating inoculums in a homogenized biofilm (A) and in liquid (B). Enlarged images of the both seeds are also shown (the scale bar is 200 μm).

### EMBODIMENTS TO PERFORM THE INVENTION

The present invention provides a seed having inoculated with a biofilm of a Bacillus bacterium. The Bacillus bacterium may be of any form, including a bacterium per se, prespore, and spore, and may be inoculated dead or alive. The species of Bacillus bacteria are not limited, including Bacillus subtilis, Bacillus anthracis and Bacillus cereus, for example. In the present invention, Bacillus subtilis is particularly preferably used, with an example of strains of Bacillus subtilis RB14. Bacillus subtilis RB14 is known as a bacillus gram positive soil bacterium that can produce lipopeptide antibiotics, iturin A and surfactin (Non-Patent Documents 3, 4 and 5).

A biofilm means a structure of a microorganism obtainable by static culture of a microorganism. While a biofilm often has a membrane-like structure, it may take other forms. Various biofilms have been reported for Bacillus subtilis (Non-Patent Documents 8, 9, 10 and 11). A biofilm is thought to be a natural form of microorganisms and microorganisms are considered to live in a biofilm in a community in extracellular polymeric substances (EPS) existing on the surfaces of a solid or liquid.

Inoculation is also called as coating, and to inoculate a seed means to make a contact of a microorganism in the form of a biofilm or any other form with a seed and allow attachment of the microorganism on the seed. Inoculation of a biofilm may be achieved by soaking a seed in a biofilm, spraying or so on. Inoculation may be performed under conditions with, for example, stirring or mixing, when necessary. Further, the temperature, pressure, and/or humidity etc. may be changed during, before or after the inoculation.

The seed may be of any kind of plant seeds without limitation, including seeds of tomatoes, rice plants, barley and wheat, maize, asparagus, soybeans, okra, pumpkins, cabbages, cucumbers, Komatsuna (Japanese mustard spinach), Japanese white radish, onions, eggplants, carrots, green onions, Chinese cabbage, parsley, bell peppers, red peppers, broccolis, spinaches, melons, and lettuces.

The seeds of the present invention are characterized in that they prevent damages due to a disease in plants. In particular, damages due to a disease related to fungi is prevented, Examples of damages due to a disease include sheath blight, phoma rot (blight), wilt, root rot, and more specifically, die back and blight, spot-producing diseases (Entomosporium leaf spot, gray leaf spot, angular leaf spot, brown circular leaf spot, cylindrosporium leaf spot, leaf spot (brown spot), leaf spot (brown leaf spot), black rot (black spot), scab (black spot), fruit spot (black leaf spot), black spot (leaf spot), leaf spot (violet leaf spot, fruit and leaf spot), anthracnose (fruit), anthracnose, white rust, leaf spot (white leaf spot), heart rot (canker), sooty blotch (leaf blot), scab, gummy stem blight, Fusarium wilt, leaf spot (leaf blight), Zonate leaf spot, leaf spot (ring spot), gray mold, stem rot, flower blight, damping-off, white root rot, bacterial wilt, rice blight, bottom rot (basal canker), Phomopsis seed decay, yellows, fruits soft rot, bud blight, gray-mold neck rot (gray bulb rot), brown rot, foot rot (stem rot), dry rot, stem canker (stem blight), scurf (Coccochorina spot), Sphaceloma scab (anthracnose, bird's eye rot), black root rot, swelling arm, small sclerotial (neck rot), Verticillium wilt, wilt (Fusarium wilt), ripe rot, canker (Cytospora canker), leaf mold, leaf blight (brown patch), brown stem rot (needle cast), bulb rot, storage diseases (blue mold, green mold, stem-end rot, anthracnose, leaf spot (black spot)), bud blight, Zonate leaf blight, black root rot, leaf spot, damping-off, wilt, wilt, powdery mildew, leaf mold, gray mold, root rot (Pythium rot), leaf blight, brown rot, and leaf spot.

Further, the present invention provides a method for inoculating a seed with a biofilm of a Bacillus bacterium, comprising a step of forming a biofilm by culturing a Bacillus bacterium and soaking a seed in the biofilm.

In such a case, the Bacillus bacterium may be of any form, including a bacterium per se, prespore, and spore, and it may or may not alive upon the inoculation. Further, the species of Bacillus bacterium is not limited, including Bacillus subtilis, Bacillus anthracis, and Bacillus cereus. In the present invention, Bacillus subtilis is particularly preferred, with an example of Bacillus subtilis strain RB14.

The present invention is aimed to achieve the prevention from damages due to a disease in a crop. In particular, it may be aimed to prevent damages due to fungal diseases. Those diseases include sheath blight, phoma rot (blight), wilt and root rot, and more specifically, die back and blight, spot-producing diseases (Entomosporium leaf spot, gray leaf spot, angular leaf spot, brown circular leaf spot, cylindrosporium leaf spot, leaf spot (brown spot), leaf spot (brown leaf spot), black rot (black spot), scab (black spot), fruit spot (black leaf spot), black spot (leaf spot), leaf spot (violet leaf spot, fruit and leaf spot), anthracnose (fruit), anthracnose, white rust, leaf spot (white leaf spot), heart rot (canker), sooty blotch (leaf blot), scab, gummy stem blight, Fusarium wilt, leaf spot (leaf blight), Zonate leaf spot, leaf spot (ring spot), gray mold, stem rot, flower blight, damping-off, white root rot, bacterial wilt, rice blight, bottom rot (basal canker), Phomopsis seed decay, yellows, fruits soft rot, bud blight, gray-mold neck rot (gray bulb rot), brown rot, foot rot (stem rot), dry rot, stem canker (stem blight), scurf (Coccochorina spot), Sphaceloma scab (anthracnose, bird's eye rot), black root rot, swelling arm, small sclerotial (neck rot), Verticillium wilt, wilt (Fusarium wilt), ripe rot, canker (Cytospora canker), leaf mold, leaf blight (brown patch), brown stem rot (needle cast), bulb rot, storage diseases (blue mold, green mold, stem-end rot, anthracnose, leaf spot (black spot)), bud blight, Zonate leaf blight, black root rot, leaf spot, damping-off, wilt, wilt, powdery mildew, leaf mold, gray mold, root rot (Pythium rot), leaf blight, brown rot, and leaf spot.

### EXAMPLES

Specific examples of the present invention are described below. The present invention, however, is not restricted to the examples.

In the examples, Bacillus subtilis was used as the Bacillus bacterium. The bacterium was cultured statically to form a biofilm, or cultured in liquid with shaking as a control to the biofilm. They were inoculated into tomato seeds and the effects of prevention against pathogen infection were observed.

As the pathogen, Rhizoctonia solani K-1 (Non-Patent Document 6) was used, which had been isolated from Kanagawa Horticultural Exp. Stn. Rhizoctonia solani is a mold known as a pathogenic microorganism of plants, which causes sheath blight, phoma rot (blight), wilt and root rot in many plant species. Particularly, characteristics of tomato damping-off have been reported in detail (Non-Patent Document 7).

### Composition of Culture Media

Bacillus subtilis RB14 was precultured in liquid shaking culture in the modified LB medium (containing 10 g/L polypepton (Nihon Pharmaceutical Inc., Tokyo, Japan), 5 g/L yeast extract (Oriental Inc., Tokyo) and 5 g/L NaCl, and adjusted to pH 7.0).

Further, the L agar plate of the modified LB medium with 1.5% agar (Shimizu Syokuhin Kaisya Ltd.) was used for determining colony forming unit (cfu).

For the main culture of Bacillus subtilis RB14, a medium containing 50 g/L fish meat proteins (provided by Kamaboko company, Odawara, Japan), 67 g/L glucose, 5 g/L KH₂PO₄, 0.5 g/L MgSO₄. 7H₂O, 25 mg/L FeSO₄. 7H₂O, 22 mg/L MnSO₄. 7H₂O and 184 mg/L CaCl₂ was used.

Rhizoctonia solani K1 was cultured in the potato dextrose broth (PDB) medium containing 200 g/L potato infusion, 20 g/L glucose and 10 g/L polypepton, and adjusted to pH 5.6.

Culture of Bacillus subtilis RB14

Five mL of frozen stock solution (stored in 10% glycerol solution at −80° C.) of Bacillus subtilis strain RB14 (Non-Patent Document 4) was added to the modified LB medium and incubated at 37° C. for 16 hours with shaking at 120 strokes per minute (spm) for preculture. A 400 μL aliquot of the overnight culture of Bacillus subtilis RB14 was used to inoculate 40 mL of the fish meat proteins medium in a 200 mL Erlenmeyer flask. Then, the flask was incubated at 25° C. for 3 days under static conditions. A biofilm was formed on the surface of the liquid medium as a membrane. Similarly, the bacterium was cultured at 30° C. for 3 days with shaking at 120 spm to obtain liquid culture. It was used as the control of the biofilm in the subsequent experiments.

Culture of R. solani K1

R. solani K1 was cultured on the PDA agar plate and an aliquot was used to inoculate 40 mL of PDB in a 200 mL Erlenmeyer flask, which was incubated at 28° C. in the dark for 7 days.

### Measurement of RB14 Attached to the Seeds

To determine the number of RB14 cells attached to the seeds inoculated with the biofilm or the liquid shaking culture, 10 inoculated seeds were taken into a sterile polypropylene tube followed by mixing for 1 minute, and the content of the tube was spread on L agar plates after serially diluted with 0.85% NaCl. The plates were incubated at 37° C. for 16 hours to count the colonies.

### Measurement of RB14 in Soil

For determining viable RB14 in soil, 3 g of soil containing RB14 was suspended in 8 mL of 0.85% NaCl solution (pH 7.0) in a 50 mL Erlenmeyer flask, shook at 150 spm for 15 minutes, and viable RB14 cells were counted using L agar plates as stated above.

### Preparation of Soil

Commercially available black soil (Nittai Inc., Tokyo, Japan) of pH 4.2 containing 10.5% total carbon and 0.6% total nitrogen was used as the soil. The soil was mixed with vermiculite at a ratio of 4:1 (w/w). The prepared soil was put in a polypropylene bag and autoclaved 3 times with 12 hours interval at 121° C. for 20 minutes. Then, the soil was improved with a fertilizer (N—0.04%, P—0.09%, K—0.06%, Ca—0.06%, Mg—0.06%, Fe—0.001%) and adjusted to about 60% of the maximum water holding capacity by adding sterile distilled water (SDW).

### Inoculation of Liquid Shaking Culture

Thirty mL of the liquid shaking culture prepared as stated above was centrifuged at 4° C. for 10 minutes at 10,000×g and the density of the bacterial cells in the culture was adjusted to about 10⁹ cfu/mL. Tomato seeds were soaked in the suspension for 15 minutes and allowed to germinate on 2% agar plates at 30° C. in the dark for 3 days.

### Inoculation of Biofilm

A biofilm at the interface was recovered from the fish meat proteins medium containing the biofilm cultured as stated above. A fresh biofilm of 2 g wet weight was mixed with 8 mL of sterile water and homogenized with the Physcotron homogenizer (NS-310E, NITI-ON Inc., Tokyo, Japan) for 15 seconds. The homogenized sample was used to coat the seeds. The bacterial cell density in the homogenized biofilm was 10⁹ cfu/mL. Tomato seeds were soaked in the inoculating material for 15 minutes and allowed to germinate as stated above. An autoclaved biofilm was prepared as XXXX.

### Preparation of Seed as Control

Tomato seeds were disinfected with 70% ethanol and then 0.5% sodium hypochlorite. The seeds were rinsed several times, and allowed to germinate as stated above.

### Plant Cultivation Experiment

Sterile soil (150 g) was placed in a plastic pot with about 90 mm diameter and 80 mm height. Germinated seeds were seeded at 9 seeds per pot and placed in a growth chamber at 30° C. with 90% relative humidity under the condition of 16 hour light (about 6,000 lux).

In an experiment using infected soil, the soil infected with a pathogenic fungus R. solani K1 strain was used. Specifically, a mycelium mat of R. solani formed on the surface of PDB was homogenized in sterile water with a homogenizer (ACE homogenizer, Nihonseiki Inc., Tokyo, Japan) at 4,000 rpm for 2 minutes, and used to inoculate the soil 6 days prior to seeding of the germinated tomato seeds. The inoculum was introduced into the soil at the dose of 1 g mycelium per 100 g soil.

### Statistic Analysis

Each plant experiment was repeated 4 times and the means of respective data were analyzed by Fisher analysis of variance.

### Measurement of Iturin A in Soil

Soil (3 g) from each pot was suspended in 21 mL of a solvent [acetonitrile and 3.8 mL trifluoroacetic acid (4:1 v/v)] in a 50 mL Erlenmeyer flask and kept at room temperature for 1 hour in a shaker (140 spm). The soil suspension was filtrated with Whatman No. 2 filter (Advantec Inc., Tokyo, Japan) and the filtrate was dried by evaporation. The pellet was dissolved in 2 mL methanol and dispensed into a 1.5 mL Eppendorf tube(s). The solution was centrifuged at 15,000×g for 2 minutes. The supernatant was filtrated with a polytetrafluoroethylene (PTFE) filter (Advantec Inc.) having 0.2 μm pore size. The filtrated solution was quantified by high-speed liquid chromatography (HPLC) using an ODS column (Chromolith Performance RP-18e 100-4.6, Merck KgaA Inc., Darmstad, Germany). For the measurement of iturin A, the system (LC-800 system, JASCO Inc., Tokyo, Japan) was run at 2.0 mL/minute and monitored at 205 nm with the eluent of acetonitrile:10 mL ammonium acetate (35:65 v/v).

### Evaluation of Fungi Inhibition on Plate

The test was performed on the potato dextrose agar (PDA) plate. A small mat of a fungal pathogen was placed at the center, followed by seeding of the seeds inoculated with a biofilm or liquid shaking culture near the center, and the plate was incubated at 28° C. for 5 days under static conditions. The inhibition of fungal proliferation was evaluated by measuring the size of the inhibition circle (mm). Further, sterilized seeds were seeded as a control. The proliferation of the test fungus was calculated as a percentage (%) by using the formula:

(a1−a2/a1)×100

wherein a1 is the proliferation of the fungus without an anti-fungal agent (calculated as an area based on the measured diameter) (negative control), and a2 is the proliferation of the fungus in the presence of RB14. The inhibition rate was obtained by deducting the measurement from 100.

### Results

### Inhibitory Activity on Plate

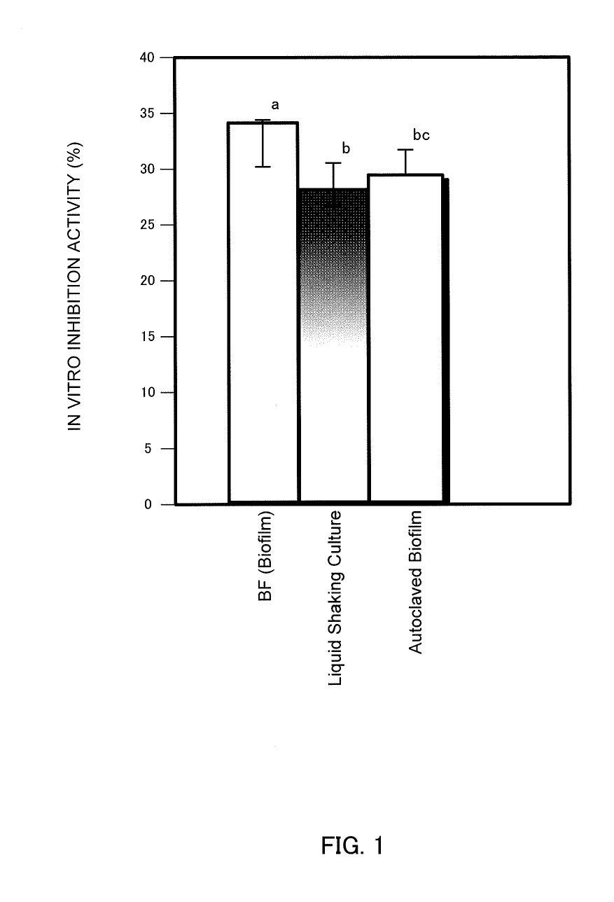

FIG. 1 shows a comparison of inhibitory activities on plates of seeds inoculated with a biofilm, liquid shaking culture or autoclaved biofilm. The seeds inoculated with a biofilm showed the highest inhibition rate (34%). The seeds inoculated with liquid shaking culture enabled pathogen inhibition of 28%. The seeds inoculated with an autoclaved biofilm showed an inhibition rate of about 30%. Control seeds did not show the inhibition activity.

### Plant Experiment

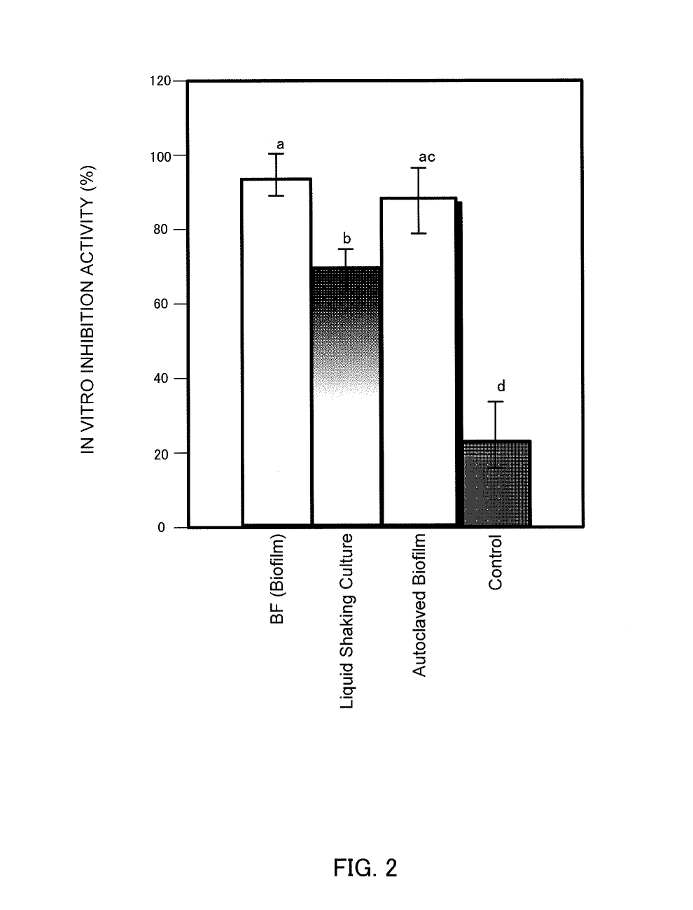

FIG. 2 shows inhibition of fungal proliferation in tomato seedlings in the plant experiment when seeds inoculated with a biofilm, liquid shaking culture or autoclaved biofilm were used on the soil infected with R. solani. When the control seeds were seeded on the soil infected with R. solani, 77% of the plants developed damping-off. The seeds inoculated with the liquid shaking culture showed damping-off in 30% of the plants, and the seeds inoculated with the biofilm showed damping-off in 6%, demonstrating remarkable reduction.

### Microscopic Analysis

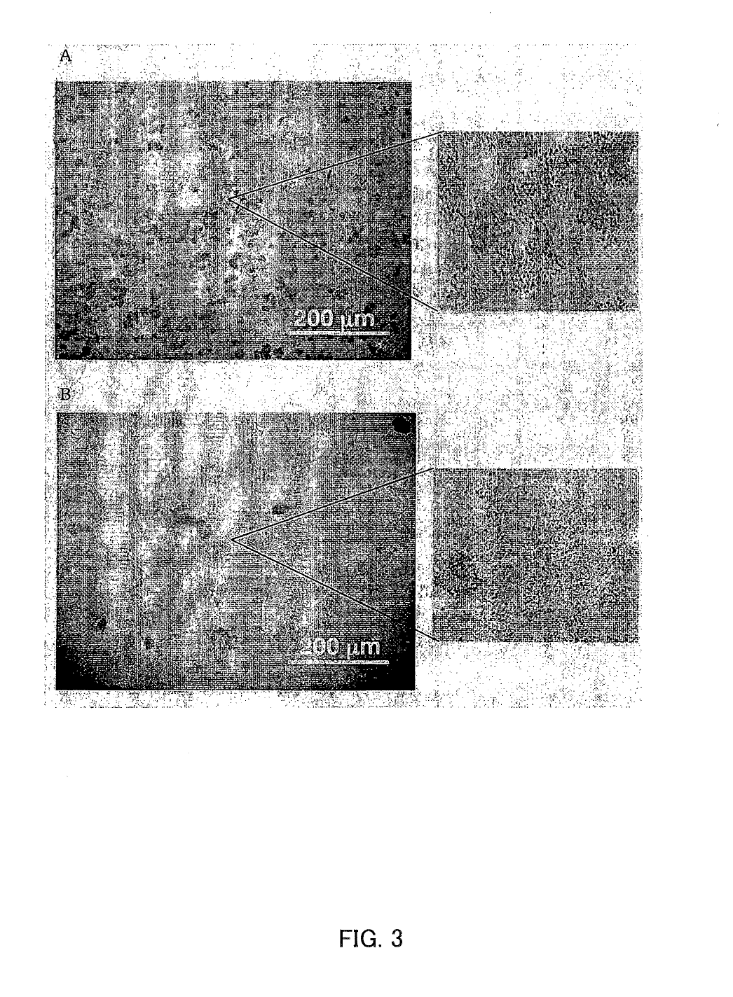

FIG. 3 shows micrograms of the surface of a seed inoculated with a biofilm or liquid shaking culture. FIG. 3A represents seeds inoculated with a biofilm, and FIG. 3B represents seeds inoculated with the liquid shaking culture. It was observed that the seeds inoculated with the biofilm contained cell aggregates whereas the seeds inoculated with the liquid culture contained mainly dispersed cells. It is speculated that these results were obtained because the biofilm contained a large amount of polysaccharides and the attachment to cells was firm.

Number of Bacillus subtilis and Iturin A concentration

Results of numbers of Bacillus subtilis and iturin A concentrations measured for seeds inoculated with a biofilm, liquid shaking culture or autoclaved biofilm are shown in Table 1.

Each seed after inoculation had around 1-2×10⁸ cells of Bacillus subtilis. After seeding the seeds, Bacillus subtilis in soil was 1-2×10⁷ cfu/g. Seeds inoculated with a biofilm had cells twice as many as the seeds coated with the liquid shaking culture, and about twice number of cells were also observed in the soil after seeding. Bacillus subtilis is known to produce iturin A, a lipopeptide antibiotics. When iturin A in the soil was measured, it did not exist in the soil before seeding, but a considerable amount was detected from the root of the tomato plant by the eventual harvesting time (Table 1).

TABLE 1

| Form of Bacillus | Bacillus | Number of Bacillus | | | |
|---|---|---|---|---|---|
| subtilis upon | subtilis | subtilis in soil | | | |
| inoculation | Cells/ | (cfu/g dry soil) | | Iturin A concentration | |
| of seeds | seed | seeding | harvesting | seeding | harvesting |
| Biofilm | 2 × 10⁸ | 0 | 2 × 10⁷ | 0 | 13 |
| Liquid shaking | 1 × 10⁸ | 0 | 1 × 10⁷ | 0 | 5 |
| culture | | | | | |
| Autoclaved | 2 × 10⁸ | 0 | 2 × 10⁷ | 0 | 10 |
| biofilm | | | | | |

Based on the results as shown above, it was shown that seeds inoculated with a biofilm of Bacillus subtilis RB14 had direct inhibitory effects against proliferation and growth of R. solani K1, thereby inhibiting damping-off of the tomato plants. This activity is speculated as being derived from EPS in the biofilm and production of lipopeptide antibiotics, particularly iturin A. The present example also shows that seeds inoculated with a biofilm of Bacillus subtilis was effective for the prevention of damages due to diseases, suggesting that similar effects against R. solani K1 are expected for other diseases, particularly those of mold-type.

This application claims for priority based on the Japanese patent application No. 2009-128816 filed on May 28, 2009, and the contents of that application is incorporated to the present application by reference.

1. A seed inoculated with a biofilm of a Bacillus bacterium.

2. The seed as stated in claim 1, wherein the Bacillus bacterium is Bacillus subtilis.

3. A method for inoculating a seed with a biofilm of a Bacillus bacterium, comprising forming a biofilm by statically culturing the Bacillus bacterium, and soaking the seed in the biofilm.

4. The method for inoculating a seed with a biofilm of a Bacillus as stated in claim 3, wherein the Bacillus bacterium is Bacillus subtilis.

5. The method for inoculating a seed with a biofilm of a Bacillus as stated in claim 3 or 4, wherein the method is carried out aiming for the prevention of damages due to a disease in a crop.
